(19) United States
(12) Patent Application Publication
(10) Pub. No.: US 2007/0122509 A1
(43) Pub. Date: May 31, 2007

(54) ANTIOXIDANT DIETARY SUPPLEMENT COMPOSITIONS AND METHODS FOR MAINTAINING HEALTHY SKIN

(75) Inventor: Piotr Chomczynski, Cincinnati, OH (US)

Correspondence Address: FROST BROWN TODD, LLC, 2200 PNC CENTER, 201 E. FIFTH STREET, CINCINNATI, OH, 45202, US

(73) Assignee: Molecular Research Center, Inc., Cincinnati, OH (US)

(21) Appl. No.: 11/559,205

(22) Filed: Nov. 13, 2006

Related U.S. Application Data
(63) Continuation-in-part of application No. 11/273,514, filed on Nov. 14, 2005.
(60) Provisional application No. 60/632,481, filed on Dec. 2, 2004.
(60) Provisional application No. 60/708,498, filed on Aug. 16, 2005.

(51) Int. Cl. A61K 36/81

(52) U.S. Cl. 424/777

(57) ABSTRACT

Compositions and methods for maintaining healthy skin and alleviating skin conditions such as redness, inflammation, irritation and skin aging, as well as for maintaining healthy scalp and hair are disclosed. The oral compositions disclosed comprise antioxidants including lycopene, gallic acid and ascorbic acid. Preferably lycopene containing tomato compositions has been water-extracted, more preferably under acid conditions. In the method of treatment aspect of the invention, an oral composition containing antioxidant(s) is administered to a person concurrently with a topical treatment for said skin conditions. Preferred topical compositions comprise cyclohexane polyols, such as cyclohexanediol or cyclohexanetriol.

### CROSS-REFERENCE TO RELATED APPLICATIONS

This application is a continuation-in-part of U.S. patent application Ser. No. 11/273,514, filed Nov. 14, 2005, which is related to and claims priority from U.S. Provisional Application Ser. No. 60/632,481, Chomczynski, filed Dec. 2, 2004, and U.S. Provisional Application Ser. No. 60/708,498, Chomczynski, filed Aug. 16, 2005, both incorporated herein by reference.

### TECHNICAL FIELD

The present invention relates to the use of antioxidant dietary supplements, alone and together with topical treatments, for maintaining healthy skin and alleviating skin conditions such as redness, inflammation, irritation and skin aging.

### BACKGROUND OF THE INVENTION

A proper diet is maintaining contributing factor in maintaining healthy skin. There are a variety of known dietary supplements affecting human skin conditions. (International Cosmetic Ingredient Dictionary and Handbook, 2004; and U.S. Pharmacopoeia Dietary Supplement Monographs).

In my previous patent application (Chomczynski, U.S. patent application Ser. No. 10/925,851, filed Aug. 25, 2004) we disclosed that oral administration of tomato products can be beneficial for maintaining healthy skin and for treatment of skin conditions, when used in conjunction with a topical treatment including cyclohexanol derivatives. It was disclosed further that an active component(s) of tomato is associated with a water insoluble tomato residue. The water insoluble tomato residue is mainly composed of plant polysaccharides forming a matrix inter-spaced with water insoluble tomato components, most notably carotenoids. There are several carotenoids that have been identified (Khachik et al., 2002) in tomato (lycopersicum esculantum and its wild variety). The most characteristic carotenoid found in tomato is lycopene. Lycopene is present in high concentration in ripe tomato fruits and also in watermelons, pink grapefruits, pink guavas and papayas.

The health benefits of lycopene-containing food products are substantial. Known beneficial health effects of lycopene include: reducing risk of certain cancers (such as prostate cancer), and protecting tissues from oxidative damage especially protecting proper function of the human eye (Khachik et al, 2002). Lycopene has been used in cosmetic compositions and oral supplements in humans (www.lycopene.org).

Lycopene has also been used as a part of cosmetic and medical compositions to improve skin health. In cosmetic compositions, Berens et al. (U.S. Published Patent Application 2004/0170585, published Sep. 2, 2004, par. 19) used lycopene as an antioxidant in the treatment of skin pigment disorders. Cochran describes compositions for providing nutrients and regulatory components to the human body; those compositions include lycopene as an antioxidant (U.S. Pat. No. 6,048,846, Cochran, issued Apr. 11, 2000).

Gupta, in U.S. Published Patent Application 2004/0161435, published Aug. 19, 2004, discloses topical treatment of skin aging promoting collagen and elastin in the skin by a treatment with a composition including lycopene.

Lycopene is included in topical compositions to treat human skin to affect tanning activity (WO 97/47278) and for treatment of the scalp and/or acne affecting 5 alpha-reductase activity (JP-2940964).

Popp et al, describes a dietary supplement composition for promoting healthy skin with lycopene as a part of composition containing other necessary ingredients such as vitamins, microelements and other chemically defined components (U.S. Pat. No. 6,630,158, issued Oct. 7, 2003). The supplement is provided in the form of a tablet, powder, capsule, wafer, liquid or liquid filled capsule.

Gorsek describes, treatment of age-related eye ailments with an orally ingested composition including lycopene (U.S. Pat. No. 6,103,756, issued Aug. 15, 2000).

Lorant et al. (U.S. Pat. No. 6,623,769, issued Sep. 23, 2003) uses lycopene in the form of a suspension or solution for oral administration and for topical application to improve cutaneous skin aging.

Winston et al. (US Patent Application 2003/0008048) describes a two-part composition containing in one part lycopene (from tomato oleoresin) and tomato powder for helping the body resist effects of aging process.

Adsorption by humans of lycopene from dietary sources can be as low as 2.5% (Tyssandier et al., 2003). Heating and processing tomatoes into tomato paste increases bioavailability of lycopene (Hof, 2000). Also, presence of oil increases bioavailability of lycopene (Deming, 1999, p. 2216).

Plant fiber can be used as a dietary supplement to obtain a beneficial effect on bowel health (U.S. Pat. No. 6,753,019, Lang et al., issued Jun. 22, 2004). The fiber described in the U.S. Pat. No. '019 patent includes water-extracted tomato fiber. The extracting fluid is most preferably water, but organic solvents such as chloroform and hexane can also be used for the extraction (p. 6). Since lycopene is soluble in both chloroform and hexane, the benefit of the fiber composition described in the U.S. Pat. No. '019 patent does not appear to be related to lycopene. In addition, it is indicated that the extracted material was not treated with either acid or alkali (claim 14).

Zelkha et al. (WO 95/16363) describes extraction of lycopene from tomato pulp using organic solvents. The extraction process yielded oleoresin-containing lycopene and the lycopene-free pulp.

Schmoutz et al (U.S. Pat. No. 6,713,100, issued Mar. 30, 2004) describes confectionery products comprising at least 25% fat, 6% to 15% sugar, and at least 15% vegetable solids. The vegetable solids can include tomato solids (claim 6).

In addition to lycopene, various antioxidants were used as an adjunct part of dietary supplement to improve skin conditions (International Cosmetic Ingredient Dictionary and Handbook, 2004; and U.S. Pharmacopoeia Dietary Supplement Monographs, Vaya J, 1).

PCT Published Patent Application WO 2006/060470, published Jun. 8, 2006, is the parent of the present application. It discloses the oral administration of antioxidants, such as lycopene, together with the topical treatment for a skin condition such as acne or rosacea.

### SUMMARY OF THE INVENTION

The present invention relates to orally administered compositions used to alleviate symptoms of skin conditions, including redness, break-outs and flare-ups often associated with acne, rosacea and inflammation. The compositions comprise tomato solids containing lycopene as a dispersion in a plant matrix. The compositions may also include a fruit-derived material, such as derived from mango, apple or pineapple.

The present invention also relates to a method of maintaining healthy skin and relieving skin conditions associated with inflammation, irritation and skin aging, comprising administering to a subject in need of such treatment a topical skin treatment at the site of said skin condition, together with the oral administration to said subject of a tomato composition comprising lycopene.

Preferred compositions for administration of lycopene comprise lycopene-containing tomato compositions, including juice, puree, paste and solids. Those compositions can comprise solids that have been subjected to extraction with water, or more preferably extracted with steam, for example, under acidic conditions. The tomato solids after extraction with water have significantly reduced tomato taste and aroma and can be used to produce lycopene-containing compositions with various taste characteristics, not limited to the tomato taste. The composition with lycopene-containing tomato solids can be used concurrently with the topical application of this invention or independently as a part of a health food or a food supplement.

The preferred topical compositions are based on the active compound 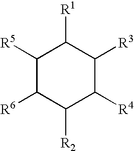
wherein R¹ is selected from —OH, and C₁-C₃ alkyl OH; and R², R³, R⁴, R⁵ and R⁶ are each independently selected from —H, —OH, COOH, C₁-C₆ alkyl and C₃-C₆ cycloalkyl; provided that the active includes no more than five —OH groups.

All patents, published patent applications, and publications discussed in this application are incorporated by reference herein.

### DETAILED DESCRIPTION OF THE INVENTION

The present invention relates to antioxidant dietary supplement compositions and methods for maintaining healthy skin, and for alleviating a number of skin conditions, including skin redness and inflammation, which are associated with skin ailments such as inflammation, irritation and skin aging. Examples of skin ailments benefiting from the present invention include acne and rosacea.

The present invention combines oral administration of medically active supplements comprising tomato compositions containing lycopene and other antioxidants with topical treatments to obtain the benefit of skin health and to treat symptoms of skin ailments.

The lycopene supplement in the current invention can further comprise synthetic and/or natural compounds or materials, known as dietary supplements, or mixtures of these compounds and materials. Examples of these compounds and materials are listed in the International Cosmetic Ingredient Dictionary and Handbook 2004, and in the U.S. Pharmacopeia Dietary Supplement Monographs, and include, for example, vitamins, microelements, natural extracts, hormones, proteins, carbohydrates, and combinations thereof.

Lycopene can be utilized in the present invention as a component of a plant-derived material. The purified lycopene has only marginal effect in the present invention.

The most preferred form of lycopene administration in the present invention is dispersion in a solid matrix. The solid matrix can be a tomato solid containing dispersed lycopene. Examples of lycopene dispersion in plant solids include: juice-containing solids, pulp, puree, mousse, sauce, paste, concentrate and powder.

The lycopene-containing tomato material can be supplemented with other antioxidants either in substantially pure form or as components of plant-derived material. Currently, the common additive to tomato juice is ascorbic acid. The other antioxidant additives in the current invention comprise hydroxylated phenols, flavonoids and polyphenols.

The effective amount of the dispersed lycopene administered daily to a person is from about 0.2 mg/kg to about 3 mg/kg of body weight. The preferred amount is from about 0.5 mg/kg to about 1.5 mg/kg, and the most preferred amount is from about 0.7 mg to about 0.9 mg per kg body weight. To consume the most preferred amount, a 70 kg person should consume daily about 49-63 mg of lycopene. This corresponds to about 524-629 ml of tomato juice or about 170-715 g of tomato paste (based on lycopene content provided by www.lycopene.org). The daily consumption of this relatively high level of lycopene-containing products may not be readily accepted due to the specific taste of tomato.

The consumption of lycopene-containing tomato products (including fresh tomato) in the United States equals about 92 pounds per capita per year, measured as fresh fruit equivalent (ers.usda briefing). This amounts to 3.5 mg of daily intake of lycopene. Tomato juice is about 15% of the total consumption of tomato products, what amounts to about 14 pound per capita, and 16 ml of tomato juice per day. By comparison, orange products have similar levels of consumption, 90 pounds per capita per year, measured as fresh fruit equivalent (ers.usda publications). However, 83% of this consumption is orange juice. This amounts to about 75 pounds or of orange juice per capita per year or 93 ml per day. The price of tomato juice is lower than orange juice and the availability of both juices is similar. One of factors limiting consumption of tomato juice is its vegetable flavor.

The most frequently consumed tomato drinks in the United States are tomato juice and various versions of V8 Vegetable Juice, a product of Campbell Soup Company (Camden, N.J.). It is a long-standing tradition to associate lycopene-containing tomato products with vegetable and salty taste. For example, most tomato juices are supplemented with salt and the V8 drinks contain tomato juice supplemented by vegetable juices including carrots, celery, beets, parsley and lettuce.

A lack of sufficient appeal of tomato juice to consumers was apparent in the survey that we conducted in the Cincinnati area (Example 11). The majority of surveyed people (74%) responded that they like tomato juice. However, only 21% of them actually drink tomato juice at least once a month. Most people in the survey (69%) drink juices other than tomato juice. Obviously, the taste of tomato juice is not sufficiently attractive to a consumer to drink it more frequently. Another tomato product, tomato paste, is currently formulated to be a part of sauces and soups and is not designed for a daily consumption of about 200 g, as required in the current invention. To approach consumption of lycopene-containing tomato products indicated in the current invention there is a need to develop new compositions with significantly improved taste.

The taste improvement of lycopene-containing tomato compositions disclosed in the current invention is accomplished by supplementation of tomato compositions with fruit flavors rather than vegetable flavors. Our tests show that fruit-flavored tomato products of this invention are preferred by consumers over products with traditional vegetable and salty taste (Examples 12 and 13). Contrary to this finding, it was assumed that tomato products should have tomato taste and color for consumer acceptance (U.S. Pat. No. 6,924,420, column 2, par. 45).

U.S. Pat. No. 6,436,452 discloses a method for improvement of tomato products by increase in fruity flavor as a result of chemical modifications. This was achieved by co-oxidation of carotenoids and polyunsaturated fatty acids added to a tomato product along with lipoxygenase. This chemical modification affected aroma of tomato product next to its taste.

The current invention discloses simple and effective methods for improving flavor, comprising taste and aroma, of tomato compositions that does not involve chemical reactions. In one approach of the current invention, lycopene-containing tomato compositions such as juice, puree and concentrate are supplemented with fruit-derived composition comprising at least 5% of that of the tomato composition, calculated per dry mass. To maintain fruity flavor, the vegetable supplement should comprise less than about 10% of the lycopene-compositions of the current invention. The fruit supplement of the current invention comprises: mango, apple, grapefruit, pineapple, cherry and berry fruits. The fruit supplement can be added to unmodified or modified tomato compositions of this invention. The modified tomato composition is defined here as a tomato product that was subjected to the taste changing-water extraction processes described in this invention. The disclosure of fruit supplement of this invention makes possible creation of new compositions with lycopene-containing tomato products with improved taste appeal to consumers.

It is disclosed in the current invention that the preferred fruit supplements to mix with tomato products derive from mango, apple and pineapple and comprise: juice, pulp, paste, powder, extract and concentrate. The most preferred fruit supplements of the current invention contain mango. Mango-derived supplements are uniquely effective in quenching the tomato flavor, comprising taste and aroma, and substantially improve the appeal of tomato products to consumers. This flavor-improving effect of mango is especially useful with tomato compositions, which were not subjected to the water extraction processes described in the current invention.

In the current invention, the minimal effective amount of mango juice or concentrate that significantly changes taste of an unmodified tomato composition is at least about 5% of the amount of the tomato composition. The preferred amount of mango is from about 10% to about 25% of the amount of unmodified tomato composition. The percent refers to the amounts of mango and tomato compositions represented as the dry mass. The tomato-mango compositions can, if desired, be supplemented by another fruit-derived component or mix of components.

It is of interest that a product of Campbell Soup Company uses mango in the name of a product “VFusion-Peach-Mango 100% Juice”. However, the product contains vegetable juice from yellow potatoes at highest concentration, followed by yellow tomatoes, carrots, white grapes, oranges, peaches and less than 2% of mango juice. Since the VFusion-Peach-Mango Juice contains only yellow tomato, it does not contain significant amount of lycopene.

The tomato-fruit compositions of the current invention can be supplemented with sugar and other components. The amount of sugar may vary in accord with the product. For tomato juice of this invention the preferred amount of sugar is from about 1 g to about 5 g per 100 g of juice. The additional components added to the tomato-fruit composition should be below 50% and preferentially lower than 10% than the amount of the fruit supplement, based on dry mass of the composition. Especially, the unique mango-tomato flavor can be easily overcome by other flavors, and most easily by vegetable flavors.

Example 12 shows taste improvement of the tomato juice of the current invention as compared with unmodified tomato juice. A group of 23 testers graded the taste of Tomato-Mango Juice of the current invention above the two unmodified tomato juices, one with high sodium and the other low sodium content.

The taste changing methods of the current invention allow for substantial reduction of sodium content in tomato products.

In my previous patent application (Chomczynski, U.S. Ser. No. 10/925,851) it was disclosed that oral administration of tomato products is beneficial for skin conditions. It was also disclosed that active ingredients of the invention were insoluble in water. In the current invention it is disclosed that the insoluble in water-active ingredients of tomato comprise lycopene. The current invention further discloses that the lycopene-containing plant solids subjected to extraction with water are preferred over the non-extracted plant solids. Surprisingly, we found that water-extracted tomato solids have significantly reduced or entirely removed characteristic flavor of tomato (comprising taste and aroma). This indicates that the component(s) contributing to the characteristic flavor of tomato is extracted from tomato using water and can be water-soluble. This is unexpected in view of the U.S. Pat. No. 6,890,574 (Geifman et al.). The U.S. Pat. No. '574 patent discloses a method for producing from the aqueous part of tomato a food taste enhancer which lacks the dominant tomato flavor (par. 50). This taste enhancer without the tomato flavor is obtained by condensation and proteolitic digestion of tomato serum, an aqueous component of tomato. Proteins do not critically contribute to flavor of a plant material. The disclosure in the U.S. Pat. No. '574 that aqueous tomato fraction is not associated with the tomato flavor is contradictory to disclosure of the current invention that water extraction of tomato solids reduces their tomato flavor.

A tomato composition, comprising paste and slurry, when subjected to water extraction described in this invention yields two fractions: a material soluble in water and a material remaining insoluble in water. The material remaining insoluble in water is named in this invention as water-insoluble solids. These water-insoluble solids are red from a high content of lycopene. Typically, the water-insoluble tomato solids retain at least 90% of lycopene present in the starting paste, slurry or other raw material. The material soluble in water comprises compounds defining tomato organoleptic properties including taste and aroma, except red color. The water-soluble fraction after condensation yields a food product additive which has the tomato taste and aroma and without, or with residual amount, of lycopene.

The tomato water-insoluble solids retain lycopene as well as beneficial effects of tomatoes on health and skin. The lack of, or significantly reduced, tomato taste of the water-insoluble tomato solids of this invention allows for manufacturing new food products containing bio-effective form of lycopene with various taste characteristics not limited to the tomato flavor.

It has further been discovered in the current invention that the most effective water extraction of plant solids is extraction with water at acidic pH. The acidic extraction should be performed at pH below about 6, and preferably at a pH range from about pH 1.5 to about pH 4.5. The pH during the acidic extraction can be adjusted, for example, with HCl. An example of extraction conditions is at ambient temperature (range about 15° C.-25° C.) and atmospheric pressure. The extraction typically takes from about 1 hour to about 12 hours. There can be a single round or multiple rounds of water and/or acidic water extraction.

Various techniques can be adapted to perform water extraction of this invention on a commercial scale including: reverse osmosis, press with one or more apertured filter screens, and sedimentation. One method of water extraction in the current invention is sedimentation. In the sedimentation method, tomato juice or a slurry obtained by mixing tomato paste or concentrate with water, is sedimented in a flow-trough centrifuge or a decanter at about 3,000 g to about 10,000 g, and preferentially at about 6,000 g. The sedimentation yields a pellet of water-insoluble solids and a supernatant containing water-soluble material. Extraction of tomato paste with 10-fold volume of water yields the water-insoluble solids that are virtually tasteless and odorless to human senses. The minimum volume of water to significantly decrease tomato flavor is about one volume. The water-insoluble tomato solids prepared by the sedimentation method using about 6,000 g retain at least 90% of lycopene present in a starting material.

The water extraction method of the current invention can be integrated into a commercial processing of tomato. This requires collecting of extracted tomato solids, mixing the solids with required fruit juice and/or components, pasteurizing the resulting mixture and packaging into containers. For a commercial large-scale application, sedimentation of tomato slurry might be performed using centrifuge with continuous discharge of solids and liquids. For example, one or line of two centrifuges can be used. Using a line of two centrifuges, the first one can be a tomato decanter such as NX438 (Tetra Pak Inc. II, USA) providing 3,000 g followed by SP-725 Superhelix Sharples centrifuge with automatic solid discharge providing up to 10,000 g (www.alafalaval.com).

The water extraction of the present invention also includes extraction with steam. In this extraction method, a steam generator provides steam that passes through a hot tomato slurry. After about 0.5 to about 3 hours of steam extraction, the tomato flavor of the slurry is significantly reduced. The remaining tomato flavor can be masked by mixing of the steam-processed slurry with food products, for example, with other fruit juices. The lycopene content of the steam-processed tomato slurry is essentially the same as the unprocessed slurry.

The simplified version of the steam extraction of the present invention comprises boiling of tomato slurry or concentrate supplemented with about 0.1 volume to about 4 volumes of water. The preferential amount of water is from about 0.5 volume to about 1 volume. The heat and steam generated during boiling sufficiently removes the tomato flavor to use the processed slurry for food products with significantly reduced tomato flavor.

The steam extraction of the present invention can be performed at about neutral pH (range 6-8), alkaline pH (range 8-12) and acidic pH (range 1-6). The preferred range of pH is about 1-6, and the more preferred range is pH about 2-3. The steam extraction of the present invention is simpler and more economical than the water extraction method based on sedimentation with the use of centrifuge.

The steam carrying the tomato aroma and taste can be condensed and the resulting condensate can be used as a food additive with a tomato taste and aroma, while colorless due to the absence of lycopene.

It has been disclosed in the present invention that an important component of the water-insoluble plant solids is lycopene and that acid-extracted plant solids are the most effective formulation providing lycopene. Purified lycopene administered in pills is only marginally effective. Dispersion of lycopene in a plant matrix can contribute to lycopene's effectiveness in the methods of treatment of the present invention. In addition to lycopene, the water-extracted plant solids retain several compounds that can contribute to health and/or skin health, for example, insoluble in water polysaccharides, glycoalkaloids and carotenoids. Carotenoids, with their antioxidative activity, and other components of water extracted tomato solids can contribute to lycopene effectiveness in the methods of treatment of the present invention. These beneficial carotenoids in tomato include compounds such as phytoene, phytofluene, carotens, neurosporene and lutein. It has been shown (Boileau, 2003) that consumption of tomato powder but not lycopene inhibited prostate carcinogenesis in rats. The authors in that paper suggested tomato products contain compounds in addition to lycopene that modify prostate carcinogenesis.

The lycopene-containing compositions of this invention can be administered as a dietary supplement in the form of paste, puree, mousse, suspension or powder. They can be administered as a part of oral/topical administration of the present invention or as stand-alone food products. The lycopene compositions of this invention may also contain components originating from more than one plant source. It may also comprise non-lycopene-containing plant solids and other plant-derived components, such as juice, extracts, powders and puree.

The tomato material processed in accord with methods described in this invention significantly loses its tomato flavor and acquires a fruity flavor. Examples 12 and 13 show substantial taste improvement of compositions of the current invention as compared with the unmodified tomato products. The unmodified tomato juices, with salt or low-salt, were considered substantially different and inferior in comparison to compositions of the current invention. In Experiment 12, except for one undecided taster in Part A and 2 undecided tasters in Part B, 20 tasters in Part A and 21 tasters in Part B classified the taste of unmodified tomato juice as substantially different and inferior when compared with Tomato-Mango Juice and Acid Extracted Tomato Grapefruit Juice of the current invention. Similarly in Experiment 13, tomato puree compositions of the current invention received a much better taste score than unmodified tomato paste. These trials show substantial improvement in taste of a composition containing tomato paste and tomato solids of the current invention compared to unmodified tomato paste. Out of 18 tasters, 13 like the taste of Tomato-Mango Puree, and 16 like Water-Extracted Tomato Grapefruit Puree. None of the tasters like consumption of unmodified tomato paste. The traditional formulation of a tomato paste is not for direct consumption. However, after its modifications according to the current invention, the resulting tomato puree is acceptable for consumption. The tomato purees of the current invention contain about 3 fold higher concentration of tomato solids and lycopene than juice and might be preferred for consumption over more voluminous and higher in calories tomato juice.

The organoleptic characteristics of the lycopene-containing compositions of the current invention can be further modified by mixing them with other components, such as juice, extracts or puree, derived from a variety of sources comprising: fruits, herbs and other food products including dairy products. The lycopene compositions of the present invention may also comprise chemical (synthetic or natural) components changing or enhancing its taste, aroma and color. The lycopene compositions of the present invention may further comprise components enriching and modifying its dietary value including, for example, protein, carbohydrate and other dietary additives as required by a diet. A diet may include consumption of the lycopene-containing compositions of the present invention alone or concurrently with the topical treatment of current invention. An example of protein enrichment of a tomato product is a composition comprising: tomato paste, mango pulp and yogurt.

The lycopene compositions of the present invention may also include components increasing the bioavailability of the lycopene. Said components include hydrophobic liquids, such as sesame oil, corn oil, avocado oil, olive oil or canola oil or mix of these oils.

The lycopene compositions of the present invention may further comprise components with a beneficial effect on health and/or skin including, for example, added antioxidants, vitamins and microelements. Currently, tomato and other fruit juices are most frequently fortified with vitamin C to improve stability and health benefits of the juice product.

Tea, coffee, chocolate and spices are known to contain various antioxidants and other ingredients beneficial for human health (Ki et al., 2003). However, rosacea sufferers should avoid drinking tea, coffee and certain spices since they induce skin inflammatory responses such as redness, breakouts and flare-ups. Tomato products have been also reported as rosacea triggers in about 25% of people surveyed (National Rosacea Society website www.rosacea.org). It is unknown what particular ingredients in the foodstuff are responsible for inducing skin inflammatory responses. However, it is disclosed in the current invention that compounds triggering rosacea present in coffee, tea and tomato are water-soluble. Thus, the water-extracted tomato solids of this invention have additional advantage for people affected by rosacea. The use of water-extracted solids containing lycopene allows for elimination from oral compositions water-soluble compounds that negatively affect health and skin conditions.

In contrast to tomato, the water extraction removes desirable components from coffee and tea, including caffeine.

The lycopene-containing compositions of the current invention for the oral administration can be supplemented with pure antioxidant or a mixture of substantially pure antioxidants. The unpurified or partially purified plant extracts or other plant-derived material containing antioxidants can also be used in the current invention. The most preferred antioxidants for use a supplements in the present invention are gallic acid, ascorbic acid and their active derivatives, and mixtures thereof. Gallic acid esters such as methyl gallate and propyl gallate are not effective in oral compositions of the current invention. Examples of other antioxidants that can be used in the current invention comprise rutin, hydroxytyrosol and oleuropein.

We have found that ingestion of relatively high quantities of antioxidants, particularly lycopene-containing compositions, ascorbic acid or gallic acid, can result in digestive system problems including excess acidity, heartburn, gastric reflux, nausea, gas or bloating. It is disclosed in the current invention that supplementing the antioxidant intake with ginger, an extract of ginger, rutin, mint, mint extracts and menthol and its derivatives or a mixture of those materials, can alleviate these problems. These materials may be administered along with the antioxidants in an amount effective to address the problem, for example, from about 2 mg to about 1000 mg/day, preferably from about 10 mg to about 250 mg/day. The materials may be formulated in a single dosage with the antioxidant, in which case they are present at from about 5 mg to about 500 mg/dosage, preferably from about 5 mg to about 100 mg/dosage.

An additional unexpected effect of the topical compositions of the current invention is their moisturizing effect on the skin. This effect benefits skin texture and reduces the appearance of wrinkles in aging skin.

In the method of treatment aspect of the present invention, the antioxidant-containing supplement is administered orally, concurrently with a topical treatment to improve the skin condition.

By “concurrent treatment” is meant that the oral supplement is administered in its recommended dosage over the same time period that the topical treatment is administered in its recommended dosage. For example, during a given two-week period, the oral lycopene (or antioxidant) supplement is administered once per day and the topical ointment is applied to a selected area of skin twice a day (for example, morning and before bed).

Any conventional topical treatment to improve skin conditions arising from acne, rosacea, skin inflammation (e.g., topical steroids), irritation and aging (e.g., alpha-hydroxy fatty acids) can be used concurrently with the oral treatment regimen of the present invention.

A preferred treatment utilizes the topical application of an active material having the following formula: 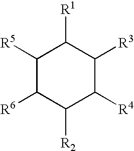
In this formula, R¹ is selected from —OH and C₁-C₃ alkyl OH(C₁-C₃ alkanols); and R², R³, R⁴, R⁵ and R⁶ are independently selected from —H, —OH, —COOH, C₁-C₆ alkyl and C₃-C₆ cycloalkyl. The total number of —OH groups in the active material should not exceed five. In this formula it is preferred that R², R³, R⁴, R⁵ and R⁶ be selected from —H and —OH, and further that the molecule in its entirety contains no more than five hydroxyl groups, and preferably no more than three hydroxyl groups. Preferred compounds for use in the present invention are selected from cyclohexanol, 2-cyclohexylethanol, cyclohexylmethanol, 3-cyclohexyl-1-propanol, 1,4-cyclohexanediol, 1,3-cyclohexanediol, 1,2-cyclohexanediol, 4-cyclohexylcyclohexanol, 4-methylcyclohexanol, 1,2,3-cyclohexanetriol, 1,3,5-cyclohexanetriol, and 1,4,5-cyclohexanetriol. Mixtures of these materials may also be used. Both the cis and trans isomers (or mixtures) of the active materials can be used herein. Stereochemical isomers and phospho- and phosphatidylo-derivatives, and metabolites of the active compounds are intended to be included within these compound definitions.

Particularly preferred compounds for use in the topical treatment aspect of the present invention include 1,2-cyclohexanediol, 1,3-cyclohexanediol, 1,4-cyclohexanediol, 1,3,5-cyclohexanetriol and 1,2,3-cyclohexanetriol, and mixtures of those materials. The cis and trans isomers, as well as the various optical isomers of these materials, are active in the present invention as well.

The active material is applied topically to the skin at the site to be treated (e.g., the site where there is skin redness or symptoms associated with, for example, rosacea, acne or skin inflammation). The active material is typically applied to the skin in an amount of from about 0.001 to about 10 mg/cm², preferably from about 0.1 to about 1 mg/cm², more preferably from about 0.1 to about 0.5 mg/cm², but this can vary depending upon the formulation, the person treated and the nature of the specific condition being treated. Maintaining healthy looking skin and relieving skin conditions, in the context of the present invention, includes maintaining healthy scalp and hair; soothing irritated skin; reducing redness, swelling and skin scars; maintaining skin texture; unplugging clogged and inflamed pores; and improving skin conditions and alleviating skin problems comprising reduction or elimination of symptoms related to skin spots, blemishes and inflammation, and improvement in appearance of aged skin. The active material may be applied in combination with a pharmaceutical or cosmetic additives and topical carriers. Topical pharmaceutical or cosmetic additives and carriers are well known in the art and are described, for example, in U.S. Pat. No. 6,723,755, Chomczynski, issued Apr. 20, 2004; U.S. Pat. No. 6,696,069, Harichian et al., issued Feb. 24, 2004; U.S. Pat. No. 6,692,754, Makimoto et al., issued Feb. 17, 2004; U.S. Pat. No. 6,660,283, Breton et al., issued Dec. 9, 2003; and U.S. Pat. No. 6,623,778, Harichian et al., issued Sep. 23, 2003; all of which are incorporated herein by reference.

When used with a topical carrier, the active material and the topical carrier together comprise a topical composition. In such topical compositions, the active material generally comprises from about 0.001% to about 10% of the composition, with the balance comprising the carrier.

### EXAMPLES

The following oral supplements are used alone or in conjunction with the topical compositions containing cyclohexanol derivatives. The topical composition is applied twice a day in the morning and evening hours to an area of skin redness. The topical composition comprises 0.5% Carbomer 940 (Spectrum Quality Products, Gardena, Calif.), 1% 1,4-cyclohexanediol (cis/trans), 0.3% glycerin and water (balance to 100%). The solution is adjusted with sodium hydroxide to pH 7.0.

The oral supplements were prepared as described in the Examples and pasteurized. When oral and topical treatments are administered concurrently, the oral supplements are consumed once a day or two times a day with meals in the morning and evening in the amount indicated below. In each example, after two weeks, the skin redness is reduced or eliminated.

### Example 1

### Tomato Juice Supplement

Tomato juice (Tomato Drink, Cinna Health Products, Molecular Research Center, Inc., Cincinnati, Ohio) containing 200 mg sodium, 2.3 g potassium, 300 mg vitamin C/liter. The Drink was supplemented with 20 mg pantothenol/liter. 250 ml of juice is consumed twice a day.

### Example 2

### Tomato Juice and Mango Supplement

Tomato juice (Tomato Drink, Example 1) was supplemented with Mango Pulp (20 g sugar/100 g, Raja Foods LLC, Skokie, Ill.) 150 g/liter. 300 ml of juice is consumed twice a day.

### Example 3

### Tomato Puree with Mango-Peach Sauce

340 g of tomato paste (Hunt's® Tomato Paste, Conagra Foods, Irvine, Calif.) is mixed with 170 g of mango-peach apple sauce (Mott's LLP, Rye Brook, N.Y.). 120 g of the resulting puree is consumed daily.

### Example 4

### Steam-Extracted Tomato Juice with Apple Cherry Juice

1000 g of Tomato Drink described in Example 1 is heated with mixing to 95 C and steam generated from 1000 g of water is passed trough the juice. After 1 h steam extraction, the juice is mixed with 500 g of Apple-Cherry juice concentrate (Old Orchard Brands, LLC, Sparta, Mich.). 300 ml of the resulting juice is consumed twice a day.

### Example 5

### Water-Extracted Supplement Mixed with Pineapple Juice

340 g of tomato paste (Hunt's® Tomato Paste) is mixed with 660 ml water and the mixture is stored at room temperature. After 6 hours of storage, the mixture is sedimented at 6 000 g and a lycopene-containing solids are again extracted with 660 ml water. The resulting water-extracted solids are suspended in 660 ml of Dole pineapple juice (Duo Juice Co., Bradenton, Fla.) and pasteurized. 200 ml of the composition is consumed once a day.

### Example 6

### Acid-Extracted Tomato Supplement Mixed with Grapefruit Juice

340 g of tomato paste (Hunt's® Tomato Paste) is mixed with 660 ml water. The mixture is acidified to pH 3 with HCl and stored at room temperature. After 6 hours of storage, the mixture is sedimented at 6 000 g and a lycopene-containing solids are again extracted with 660 ml water at pH 3. The resulting acid-extracted solids are suspended in 660 ml of Ruby Red grapefruit juice (Citrus World, Inc., Lake Wales, Fla.) containing 300 mg/liter vitamin C and 0.4 mg thiamine/liter. The pH of the mixture is adjusted to pH 4 with sodium hydroxide followed by the addition of 1.5 ml of corn oil (ACH Dietary Companies, Inc., Memphis, Tenn.). The resulting composition is pasteurized and 250 ml is consumed once a day.

### Example 7

### Acid-Extracted Tomato Supplement Mixed with Apple Sauce

340 g of tomato paste (Hunt's® Tomato Paste) is mixed with 660 ml water. The mixture is acidified to pH 3 with HCl and stored at room temperature. After 6 hours of storage, the mixture is sedimented and a lycopene-containing solids are again extracted with 660 ml water at pH 3. The resulting acid-extracted solids are mixed with 450 g of apple sauce (Mott's, LLP) containing 48 mg vitamin C. The pH of the mixture is adjusted to pH 4 with sodium hydroxide. 90 g of the mixture is consumed once a day.

### Example 8

### Acid-Extracted Tomato/Apple Supplement Fortified with Vitamins

340 g of tomato paste (Hunt's® Tomato Paste) is mixed with 660 ml water. After 6 hours of storage, the mixture is sedimented and a lycopene-containing solids are again extracted with 660 ml water at pH 3. The resulting acid-extracted solids are suspended in 450 g of apple puree. The pH of the mixture is adjusted to pH 4 with sodium hydroxide. 90 g of the mixture is mixed with a crushed multivitamin pill (CENTRUM®, Wyeth Consumer Healthcare, Madison, N.J.) and consumed once a day. The multivitamin pill contains vitamin A 3500 IU, vitamin C 60 mg, vitamin D 400 IU, vitamin E 45 IU, vitamin K 0.01 mg, thiamin 1.5 mg, riboflavin 1.7 mg, niacin 20 mg, vitamin B6 3 mg, folic acid 0.4 mg, vitamin B 12 0.025 mg, biotin 0.03 mg, pantothenic acid 10 mg, calcium 0.2 g, phosphorus 48 mg, iodine 0.15 mg, magnesium 0.1 g, zinc 15 mg, selenium 0.02 mg, copper 2 mg, chromium 0.15 mg, molybdenum 0.075 mg, chloride 0.075 mg, potassium 80 mg, boron 015 mg, nickel 0.005 mg, silicon 2 mg, vanadium 0.01 mg, lutein 0.25 mg and lycopene 0.3 mg.

### Example 9

### Lycopene Pill Supplement

Lycopene pills (Nature's Bounty, Inc., Bohemia, N.Y.), each containing 5 mg lycopene dissolved in soybean oil, are administered orally (5 pills consumed once a day).

### Example 10

### Vitamin C Pill Supplemented with Ginger

0.55 g of calcium ascorbate (Spectrum Quality Products, Inc.) and 50 mg of ginger powder is enclosed in a vegetable capsule (Capsuline, Fla.). Two pills are consumed along with tomato juice daily before breakfast and dinner.

These lycopene and antioxidant supplements may be taken concurrently with a topical skin treatment, such as cyclohexanediol, to improve acne, rosacea, skin inflammation or irritation in a patient.

Further, the supplements defined above may contain from about 0.5 mg to about 10 mg ginger extract to alleviate stomach discomfort that may accompany ingestion of high levels of antioxidants.

### Example 11

### Drinking of Tomato Juice Survey

Tomato juice survey was conducted in the Cincinnati area. 169 participants were surveyed, 83 men and 86 women. The following answers to questions were recorded.

1. Do you like tomato juice? Yes-125 (74%), No-44 (26%

2. Do you drink tomato juice at least once a month? Yes-36 (21%) No-133 (79%)

3. Do you drink other than tomato juice at least once a month? Yes-116 (69%), No-53, (31%

Number of participants: 169 (100%).

### Example 12

### Tasting of Tomato Juices

Taste trials were conducted to evaluate taste of tomato juice compositions of the current invention. The trials involved taste comparison of two compositions of the current invention with a high-salt tomato juice from a leading brand in USA (part A) and low salt-tomato juice produced for this test (part B):

Part A. The following compositions were tested:

1. Leading brand of tomato juice was from a US company that contained: sodium 4.76 g, potassium 3.0 g, sugars 29 g per liter,

2. Tomato-Mango Juice Drink described in Example 2,

3. Acid-Extracted Tomato-Grapefruit juice described in Example 6 (made of acidic-water extracted tomato solids).

Part B. The following compositions were tested:

1. Low-salt (0.2 g/l) Tomato Drink described in Example 1,

2. Tomato-Mango Juice Drink described in Example 2,

3. Acid-Extracted Tomato-Grapefruit juice described in Example 6 (made of acidic-water extracted tomato solids).

21 tasters in Part A and 23 tasters in Part B evaluated taste of products and answered the following questions:

1. Which of the three products is substantially different and inferior?

2. Which of the remaining products you prefer?

Tables below show how the taster evaluated each of the products in response to these two questions.

Part A.

| | | Question 1 | | |
|---|---|---|---|---|
| | | (different | Question 2 | — |
| | Taste | and inferior) | (preferred) | undecided |
| 1. | Unmodified, salty | 20 | 0 | 1 |
| | Tomato Juice | | | |
| 2. | Tomato-Mango Juice | 0 | 5 | 0 |
| 3. | Acid-Extracted Tomato- | 0 | 15 | 0 |
| | Grapefruit Juice | | | |

Part B.

| | | Question 1 | | |
|---|---|---|---|---|
| | | (different | Question 2 | — |
| | Taste | and inferior) | (preferred) | undecided |
| 1. | Unmodified, low-salt- | 21 | 0 | 2 |
| | Tomato Juice | | | |
| 4. | Tomato-Mango Juice | 0 | 6 | 0 |
| 5. | Acid-Extracted Tomato- | 0 | 15 | 0 |
| | Grapefruit Juice | | | |

### Example 13

### Tasting of Tomato Purees

A tasting trial of tomato purees was conducted with 18 tasters. The trial involved evaluation of the taste of unmodified Hunt's paste and two tomato puree compositions of the current invention: 1) Tomato-Mango-Peach Puree described in Example 3, made of Hunt's Paste mixed with mango peach sauce, and 2) Tomato-Apple Puree described in Example 7, made of acidic water-extracted tomato paste mixed with apple sauce. Numbers in the Table show how many tasters assigned the taste of each product to one of 3 categories: 1) I do not like it, 2) uncertain, and 3) 1 like it.

| | | do not | | like |
|---|---|---|---|---|
| | Taste | like it | uncertain | it |
| 1. | Unmodified Tomato Paste | 18 | 0 | 0 |
| 2. | Tomato-Mango-Peach Puree | 1 | 4 | 13 |
| 3. | Extracted Tomato-Grapefruit Puree | 1 | 1 | 16 |

### REFERENCES

Boileau T. W. M, et al. Prostate Carcinogenesis in N-methyl-N-nitrosourea (NMU)-Testosterone-Treated Rats Fed Tomato Powder, Lycopene, or Energy-Restricted Diets, J Natl Canc Inst 95, 1578-1586, 2003.
Deming, D. M. and Erdman, J. W., Mammalian Carotenoid Absorption and Metabolism, Pure Appl Chem, 71, 2213-2223.
Hof, K. H. et al., Carotenoid Bioavailability in Humans from Tomatoes Processed in Different Ways Determined from the Carotenoid Response in the Triglyceride-Rich Lipoprotein Fraction of Plasma after Single Consumption and in Plasma after Four Days of Consumption, J. Nutr, 2000, 130, 1189-1196.
Khachik, F. et al., Chemistry, Distribution, and Metabolism of Tomato Carotenoids and Their Impact on Human Health, Exp Biol Med 2002, 227, 845-851.
Ki et al., Cocoa Has More Phenolic Phytochemicals and a Higher Antioxidant Capacity than Teas and Red Wine, J. Agric. Food Chem., 51(25):7292-7295 (2003).
Tyssandier, V. et. al., Processing of Vegetable-Borne Carotenoids in the Human Stomach and Duodenum, A. J. Pyhysiol Gastroinest Liver Physiol, 284, G913-G923.
H.J. Heinz Company, sponsor, www.lycopene.org
National Rosacea Society, www.rosacea.org.
ERS. USDA Briefing, http://usda.mannlib.cornell.edu/reports/erssor/specialty/vgs-bb/2000/vgs282.pdf
ERS.USDA Publications, www.ers.usda.gov/publications/fts/aug03/fts30501/

What is claimed is:

1. A composition for oral consumption comprising lycopene-containing tomato: components remaining insoluble after extracting tomato material with water.

2. A composition according to claim 1 wherein said water extraction is performed at pH below about 6.

3. A composition according to claim 3 wherein said water extraction is performed at pH from about 1.5 to about 4.5.

4. A composition according to claim 1 wherein said water extraction is performed using steam.

5. A composition according to claim 1 wherein said lycopene-containing compositions are substantially reduced or free of the flavor characteristics of the raw tomato material.

6. A composition according to claim 1 further comprising non-lycopene plant material.

7. A composition according to claim 1 additionally comprising a fruit-derived material selected from juices, purees, pastes and powers, wherein the fruit-derived material comprises from about 5% to about 80% of the total dry mass of the final composition.

8. A composition according to claim 7 wherein fruit-derived material is selected from mango-derived, apple-derived, and pineapple-derived materials, or mixture of thereof.

9. A composition according to claim 7 further comprising adjunct materials selected from vitamins, antioxidants, microelements, natural extracts, dietary additives, hormones, proteins, fatty acids, oils, alcohol, carbohydrates, and mixtures thereof.

10. A composition according to claim 1 additionally comprising substantially pure antioxidant and an effective amount of a material selected from ginger or ginger extract, mint, mint extracts, mint oil and menthol and mixtures thereof.

11. A composition according to claim 10 wherein antioxidant is selected from gallic acid, ascorbic acid, pharmaceutically active derivatives of gallic acid and ascorbic acid, and mixtures thereof.

12. A method for processing tomato material to obtain lycopene dispersed in tomato solids, wherein the said solids are substantially reduced or free of the flavor characteristics of the raw tomato material, comprising water extraction of a tomato material.

13. A method according to claim 12 comprising water extraction of a tomato material using a process selected from sedimentation, press and/or reverse osmosis.

14. A method according to claim 12 comprising steam extraction of the tomato material.

15. A method according to claim 12 wherein the said extraction is performed at pH below about 6.

16. A method according to claim 15 wherein the said extraction is performed at pH of from about 1.5 to about 4.5.

17. A method of maintaining healthy skin and relieving skin conditions including conditions associated with acne, rosacea, inflammation, irritation and skin aging, comprising administering to a person in need of such treatment (a) topical treatment for said conditions at the site of said skin condition, together with (b) oral administration to said person of a composition comprising an effective amount of one or more antioxidants.

18. A method according to claim 18 wherein an effective amount of ginger, ginger extract, mint, mint extracts, mint oil and menthol is additionally administered to said person.

19. A method according to claim 7 wherein topical treatment comprises topically applying to said person, at the site of said condition, a safe and effective amount of the compound having the following formula 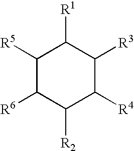
wherein R¹ is selected from —OH, and C₁-C₃ alkyl OH; and R², R³, R⁴, R⁵ and R⁶ are each independently selected from —H, —OH, —COOH, C₁-C₆ alkyl and C₃-C₆ cycloalkyl; provided that the compound includes no more than five —OH groups.

20. A method according to claim 19 wherein the compound is applied to the skin in an amount of from about 0.001 to about 10 mg/cm².

21. A method according to claim 19 wherein the compound is applied to the skin in a topical carrier.

22. A method according to claim 19 wherein the compound is selected from cyclohexanol, 2-cyclohexylethanol, cyclohexylmethanol, 3-cyclohexyl-1-propanol, 1,4-cyclohexanediol, 1,3 cyclohexanediol, 1,2-cyclohexanediol, 4-cyclohexylcyclohexanol, 4-methylcyclohexanol, 1,2,3-cyclohexanetriol, 1,3,5-cyclohexanetriol, 1,4,5-cyclohexanetriol, and mixtures thereof.

23. A method according to claim 22 herein the compound is selected from 1,2-cyclohexanediol, 1,3-cyclohexanediol, 1,4-cyclohexanediol, 1,2,3-cyclohexanetriol, 1,3,5-cyclohexanetriol, and mixtures thereof.

24. A method according to claim 19 wherein the topical composition comprises adjunct materials selected from vitamins, antioxidants, microelements, natural extract, plant materials, hormones, proteins, fatty acids, carbohydrates, oil, and mixtures thereof.

25. A method according to claim 19 wherein the oral composition comprises a lycopene-containing material extracted with water.

26. A method according to claim 25 wherein the water extraction is performed at pH below about 6.

27. A method according to claim 26 wherein the water extraction is performed at a pH from about 1.5 to about 4.

28. A method according to claim 25 where the water extraction is performed using steam.

29. A method according to claim 25 wherein the oral composition further comprises non-lycopene plant derived material.

30. A method according to claim 25 wherein the oral composition comprises lycopene-containing tomato composition and a fruit-derived composition, wherein the composition contains from about 5% to about 80% of the fruit-derived composition, measured as total dry mass of the mixture.

31. A method of claim 25 wherein an effective amount of ginger or ginger extract is additionally administered to said person.

32. A method according to claim 30 wherein the oral composition comprises materials selected from vitamins, antioxidants, microelements, natural extract, plant materials, dietary additives, hormones, proteins, fatty acids, carbohydrates, oil, and mixtures thereof.

33. A method of claim 32 that includes an antioxidant selected from gallic acid, ascorbic acid, pharmaceutically active derivatives of gallic acid and ascorbic acid, and mixtures thereof.

34. A method of claim 33 wherein the antioxidant is administered at the daily dose of from about 1 mg to about 200 mg per kg of body weight.

35. A method of claim 34 wherein the antioxidant is selected from ascorbic acid and its derivatives, and is administered in a daily dose ranging from about 3 mg to about 200 mg per kg of body weight.

36. A composition for oral consumption comprising lycopene-containing tomato components and a mango-derived component, wherein the mango component is present at least about 5% of the tomato component calculated as dry mass.
